(19) United States
(12) Patent Application Publication
(10) Pub. No.: US 2022/0079743 A1
(43) Pub. Date: Mar. 17, 2022

(54) SHAPED CORNEAL SEGMENTS: CORNEAL ALLOGENIC INTRA-STROMAL DEVICES (RING SEGMENTS AND RINGS, MODIFIED DISCS, MODIFICATIONS) FOR INDUCING SHAPE CHANGE, REGULARIZATION AND STABILIZATION OF CORNEA IN CORNEAL ECTASIA AND OTHER CORNEAL CONDITIOINS AND FOR CORRECTION OF REFRACTIVE ERRORS

(71) Applicant: SOOSAN JACOB, CHENNAI (IN)

(72) Inventor: SOOSAN JACOB, CHENNAI (IN)

(21) Appl. No.: 16/499,232

(22) PCT Filed: May 1, 2018
(86) PCT No.: PCT/IN2018/000026

§ 371 Date: Sep. 27, 2019

(30) Foreign Application Priority Data
May 2, 2017 (IN) 201741015449

(51) Int. Cl. A61F 2/14; A61L 27/24

(52) U.S. Cl. CPC A61F 2/147; A61L 27/24

(57) ABSTRACT

A device for implantation into the cornea intra-stromally comprising allogenic comeal or scleral material or other bioengineered material including, but not limited to, processed collagen tissues, comprises a segment that is inserted into a comeal channel whereby the segment regularizes the conical cornea, gives an improved surface, improves biomechanical strength distribution and stability improves optical functionality, and improves/corrects the refractive error or gives other desired shape change effects.

### FIELD OF THE INVENTION

The field of this invention refers to a device used in surgery on the human eye, in cases of corneal ectatic disorders such as keratoconus, pellucid marginal degeneration, post LASIK ectasia etc or other causes of irregular cornea. It may also be used for the treatment of refractive errors or to induce other desired shape change effects on the cornea. More specifically, the present invention relates to an allogenic device (corneal or scleral material) or made of other bioengineered material including but not limited to processed collagen that is implanted in the cornea surgically to improve the intra-operative ease, post-operative outcomes, optical functionality, regularity of cornea, corneal surface, refractive error, biomechanical strength distribution and long term stability of the eye or give other desired shape change effects.

### BACKGROUND OF THE INVENTION

There are numerous corneal ectatic conditions such as keratoconus, keratoglobus, pellucid marginal degeneration, post LASIK ectasia etc. These and other corneal conditions such as scarred corneas or post-operative corneal irregularities are associated with an irregular corneal surface and/or progressively weakening corneal biomechanics. To overcome this problem, various techniques have been described including optical correction (glasses, rigid contact lenses); implantation of artificial intrastromal ring segments; phakic intra-ocular lenses; collagen cross linking of the cornea and keratoplasty.

Refractive errors which includes myopia, hypermetropia, astigmatism, irregular astigmatism and presbyopia result in a blurred image falling on the retina causing difficulty in focus and inability to see clearly. Synthetic intrastromal corneal ring segment devices have been used to correct myopia and are implanted in the deep corneal stroma to modify corneal curvature. These were also then used for the first time to treat keratoconus and other ectatic conditions by Colin et al. These synthetic segments are made of polymethylmethacrylate and are of five main types: Intacs, Kerarings, Ferrara rings, Bisantis segments and the Myoring. These have the advantages of regularizing corneal topography, redistributing corneal stress forces, decreasing refractive error, sparing the optic zone and being reversible and adjustable. However, the patient needs to have a corneal thickness of at least 400 microns in the zone of implantation. Also, being composed of synthetic material, implantation of these artificial segments may be associated with segment migration, over-riding, focal edema around segments, stromal thinning, corneal melt and necrosis, exposure and extrusion of the segment etc especially with shallow implantation or with constant eye rubbing etc. Artificial segments can also be associated with an increased risk of infectious keratitis which can be sight-threatening. Other more innocuous complications reported include corneal neovascularization, mild channel deposits, corneal haze etc. In addition though corneal discs have been implanted to correct keratoconus by Ganesh et al, however specific modifications have not been made to fine tune and customize the disc to the patient's specific wavefront or topographic aberration pattern using laser or to increase special biomechanical properties of the disc before implantation. Implanting without these specific modifications can lead to sub-optimal visual and biomechanical results and can increase aberration patterns. In addition, bioengineered material such as processed collagen has not been used.

There is therefore a recognized need in the art for an improved device implantable in the eye which can reduce the complications that can be associated with the use of synthetic tissue or non-customized allogenic (corneal or scleral tissue disc within the eye. The present invention fulfills this long standing need in the art. By utilizing allogenic corneal or scleral material or other bioengineered material including but not limited to processed collagen as well as modifications of these, the present invention makes an outstanding contribution to technology.

### SUMMARY OF THE INVENTION

An object of the present invention is to improve surface irregularity, improve refractive error, provide desired shape changes and improve biomechanical strength of cornea in an eye of a subject following its insertion into intra-stromal corneal channels in the eye of the subject.

Improved corneal irregularity, improved surface, better biomechanical strength distribution, improved optical functionality, improved refraction, customization, improved stability, ability to induce desired shape changes, ability to implant even in corneas with less than 400 microns thickness in the zone of implantation, ability to implant at varying depths, less chances for complications such as segment migration; over-riding; stromal thinning, corneal melt, necrosis and infection; exposure and extrusion of the segment; channel deposits, corneal haze are other objects as well as advantages of this device.

### BRIEF DESCRIPTION OF THE DRAWINGS

Referring now to the drawings wherein the showings are for the purpose of illustrating preferred embodiment of the inventions only, and not for the purpose of limiting the same.

FIG. 1A: The design of an allogenic or bioengineered segment device is shown. Different parts of the segment are labelled (a—ends of the segment; b—body of the segment).

FIG. 1B: The segment is seen with reference to a human eye. The diagram shows the human cornea (a). The segment is seen ready for implantation (b). A channel (c) has been prepared either manually or using a mechanized instrument or the femtosecond laser or any other means in the cornea of the subject (a).

FIG. 1C: One segment (b1) is implanted into the channel (c) in the cornea of the subject (a). It now lies within the cornea inducing shape change, regularizing the conical cornea, giving an improved surface, improving biomechanical strength distribution and stability, improving optical functionality and improving/correcting the refractive error.

FIG. 1D: Another segment (b2) has been implanted in the opposite axis within the channel (c). Both segments (b1, b2) now lie within the cornea (a) inducing shape change, regularizing the conical cornea, giving an improved surface, improving biomechanical strength distribution and stability, improving optical functionality and improving/correcting the refractive error.

FIG. 2A: The design of an allogenic or bioengineered elongated segment device is shown. Different parts of the segment are labelled (a—ends of the segment; b—body of the segment).

FIG. 2B: The elongated segment is seen with reference to a human eye. The diagram shows the human cornea (a). The segment is seen ready for implantation (b). A channel (c) has been prepared either manually or using a mechanized instrument or the femtosecond laser or any other means in the cornea of the subject (a).

FIG. 2C: The elongated segment (b) is implanted into the channel (c) in the cornea (a). It now lies within the cornea inducing shape change, regularizing the conical cornea, giving an improved surface, improving biomechanical strength distribution and stability, improving optical functionality and improving/correcting the refractive error.

FIG. 3A: The design of an allogenic or bioengineered ring device is shown.

FIG. 3B: The ring is seen with reference to a human eye. The diagram shows the human cornea (a). The ring is seen ready for implantation (b). The plane for implantation (c) has been prepared either manually or using a mechanized instrument or the femtosecond laser or any other means in the cornea of the subject (a).

FIG. 3C: The ring (b) is implanted into the plane (c) in the cornea (a). It now lies within the cornea inducing shape change, regularizing the conical cornea, giving an improved surface, improving biomechanical strength distribution and stability, improving optical functionality and improving/correcting the refractive error.

FIG. 4A: The design of a modified allogenic or bioengineered disc device (a) is shown. Modified parts of the disc are labelled (b—modified area of the disc).

FIG. 4B: The disc is seen with reference to a human eye. The diagram shows the human cornea (a). The disc is seen ready for implantation (b). The plane for implantation (c) has been prepared either manually or using a mechanized instrument or the femtosecond laser or any other means in the cornea of the subject (a):

FIG. 4C: The disc (b) is implanted into the plane (c) in the cornea (a). It now lies within the cornea inducing shape change, regularizing the conical cornea, giving an improved surface, improving biomechanical strength distribution and stability, improving optical functionality and improving/correcting the refractive error.

FIG. 5A: The segment device (a1), elongated segment device (a2), ring device (a3), modified disc device (a4) or other variations in shape, thickness, size, length, diameter, or cross-sectional architecture of these are processed (b) to modify for shape, regional thickness variability, strength, rigidity, stiffness, safety, storage capabilities, drug elution or vector capabilities etc using a variety of different ways including but not confined to collagen crosslinking, chemical/thermal/biological/laser modification, gamma radiation, cryopreservation and so on.

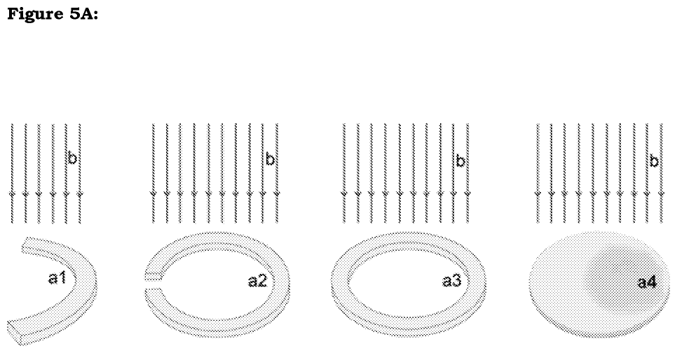

FIG. 5B: The processed segment device (b1), elongated segment device (b2), ring device (b3), modified disc device (b4) or other variations of these are implanted into the cornea (a). It now lies within the corneal channel/plane (c) inducing shape change, regularizing the conical cornea, giving an improved surface, improving biomechanical strength distribution and stability, improving optical functionality and improving/correcting the refractive error.

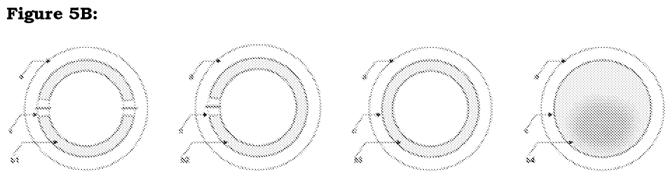

### DETAILED DESCRIPTION OF THE INVENTION

“subject” as mentioned herein ‘any recipient of the corneal allogenic intra-stromal ring/segment devices described herein’

The present invention will be described herein below with reference to the accompanying drawings. The present invention provides induction of shape change, conical regularization, improved surface, optical improvement, refractive correction and biomechanical stability for use in corneal surgery in a subject.

### Embodiment 1: Corneal Allogenic Intra-Stromal Ring Segment Design 1

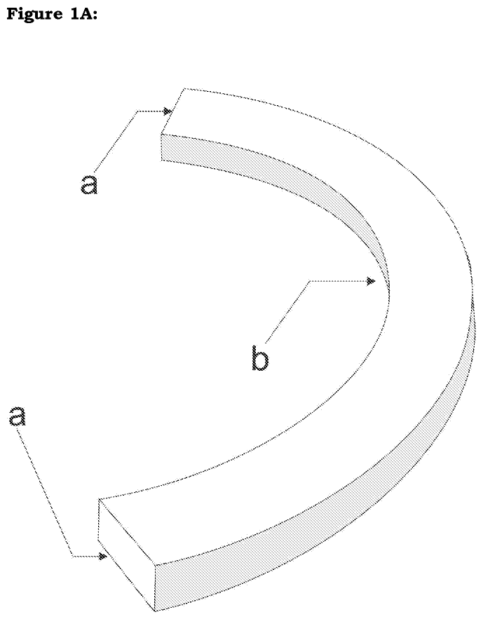

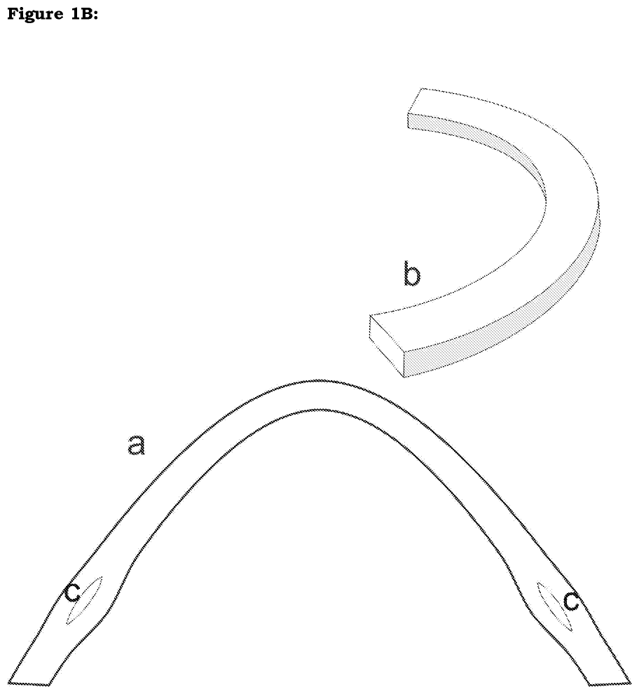

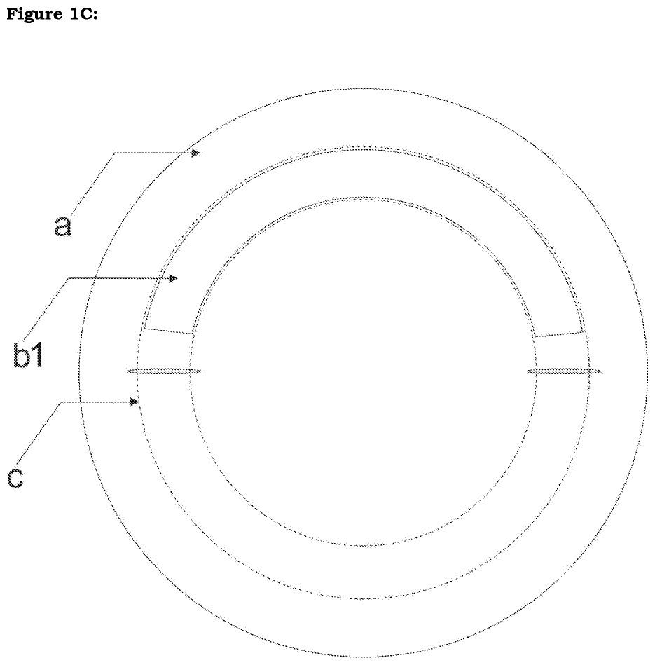

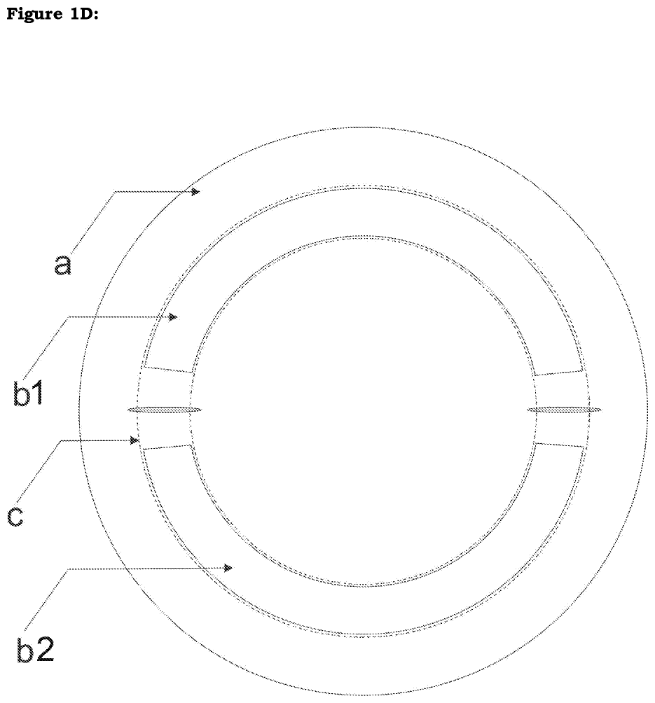

The device is made of allogenic corneal or scleral material or other bioengineered material including but not limited to processed collagen (FIG. 1A). A ring segment of this tissue is created either manually or with a special trephine or femtosecond laser or any other means. It is then implanted into the eye. The said segment may be of varying geometry, varying thickness in any dimension, varying cross-sectional architecture, varying sizes in any dimension, varying radius and diameter and varying arc and/or chord length.

### Embodiment 2: Corneal Allogenic Intra-Stromal Ring Segment Design 2

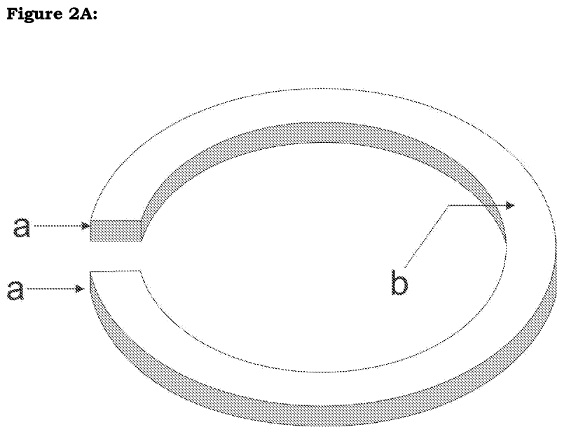

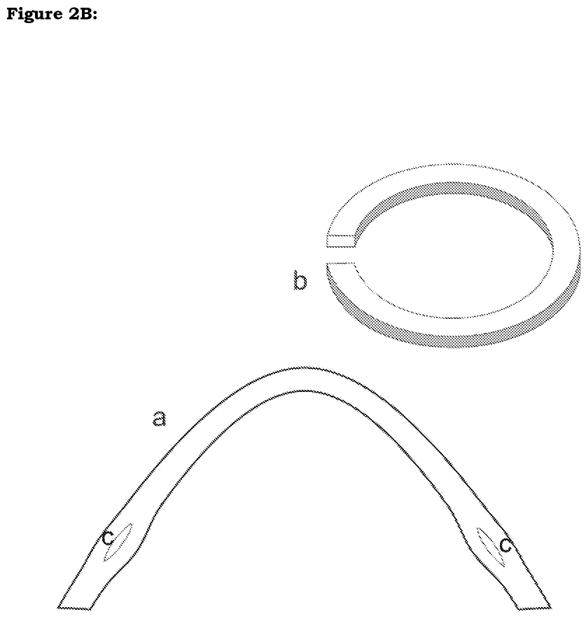

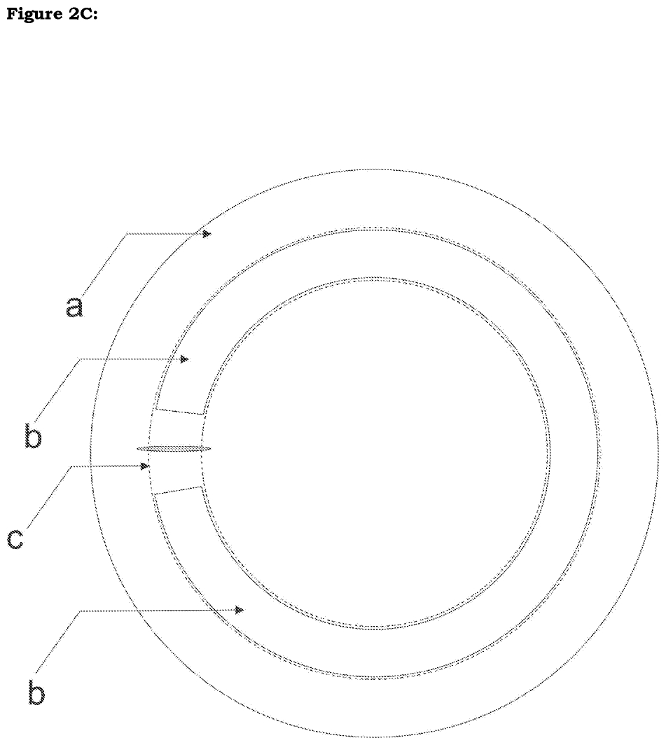

The device is made of allogenic corneal or scleral material or other bioengineered material including but not limited to processed collagen (FIG. 2A). An elongated ring segment of this tissue is created either manually or with a special trephine or femtosecond laser or any other means. It is then implanted into the eye. The said segment may be of varying geometry, varying thickness in any dimension, varying cross-sectional architecture, varying sizes in any dimension, varying radius and diameter and varying arc and/or chord length.

### Embodiment 3: Corneal Allogenic Intra-Stromal Ring Design 3

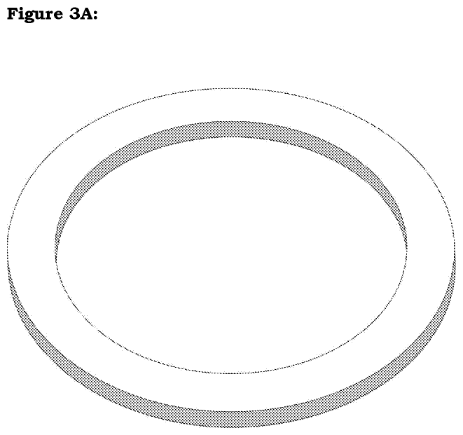

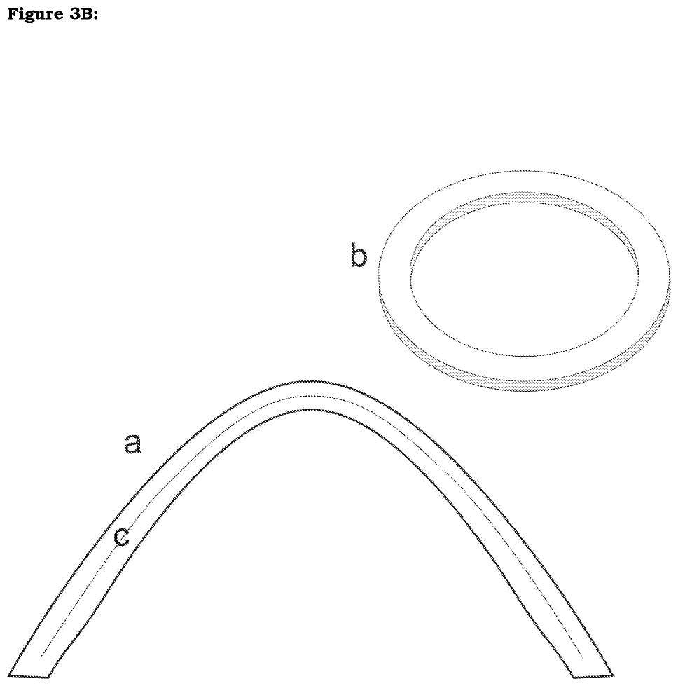

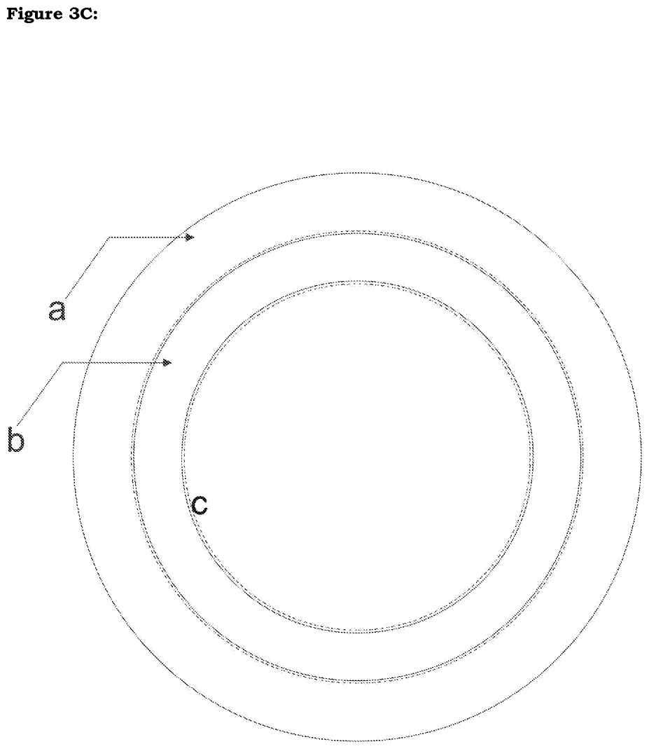

The device is made of allogenic corneal or scleral material or other bioengineered material including but not limited to processed collagen (FIG. 3A). A ring shaped device of this tissue is created either manually or with a special trephine or femtosecond laser or any other means. It is then implanted into the eye. The said segment may be of varying geometry, varying thickness in any dimension, varying cross-sectional architecture, varying sizes in any dimension, varying radius and diameter and varying arc and/or chord length.

### Embodiment 4: Modified Corneal Allogenic Intra-Stromal Devices (Modified Disc Segments)

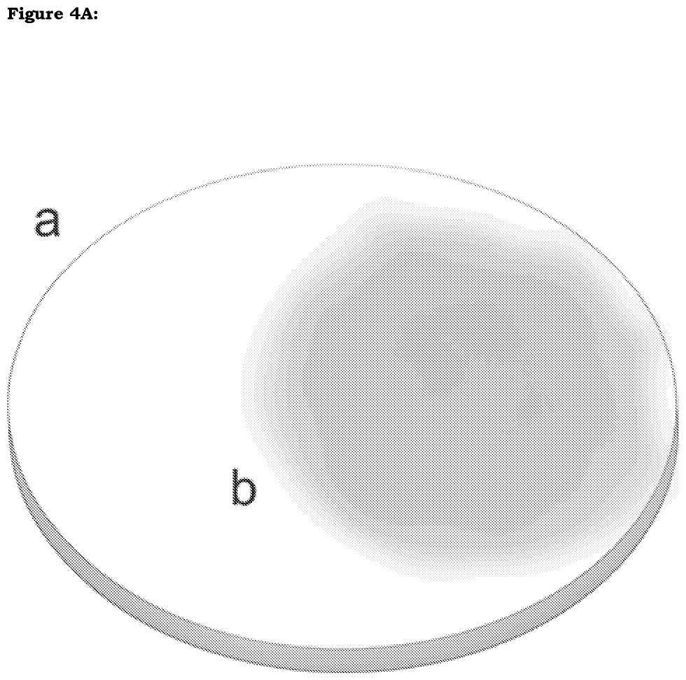

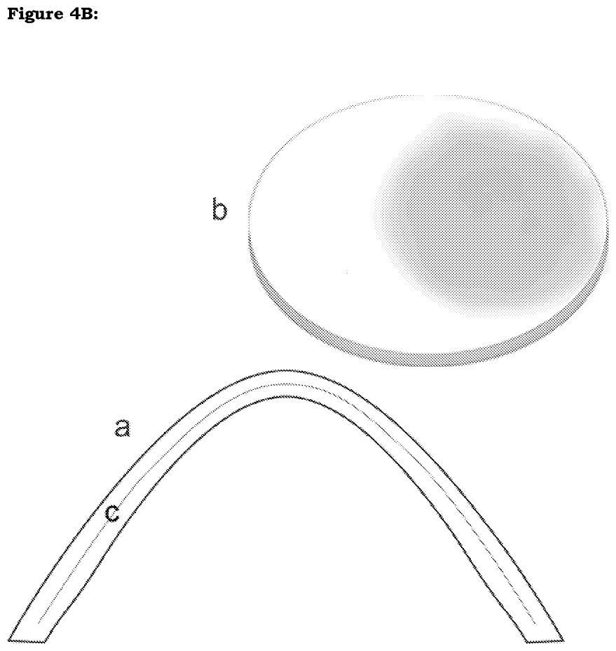

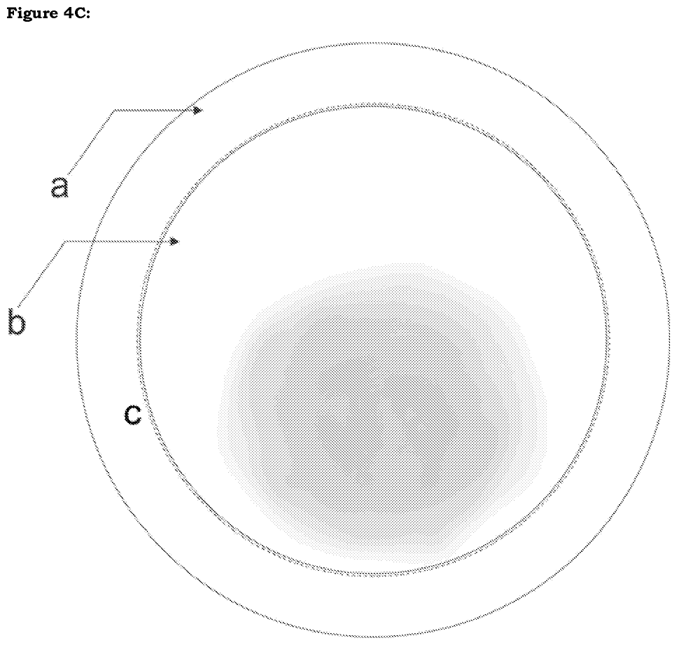

The device is made of allogenic corneal or scleral material or other bioengineered material including but not limited to processed collagen (FIG. 4A). A disc shaped device of this tissue is created either manually or with a special trephine or femtosecond laser or any other means. It is then implanted into the eye. The modified disc has regional variations in its geometry or thickness. The said disc may be of varying geometry, varying thickness in any dimension, varying cross-sectional architecture, varying sizes in any dimension and varying radius and diameter.

### Embodiment 5: Modified Corneal Allogenic Intra-Stromal Devices (Ring Segments, Rings and Modified Discs)

The device is made of different varieties of allogenic corneal or scleral material or other bioengineered material including but not limited to processed collagen. The intra-stromal device (ring segments, rings and modified discs) is further modified for shape, regional thickness variability, strength, stiffness, rigidity, safety, storage capabilities, drug elution or vector capabilities etc. This may be possible through a variety of different ways including but not confined to collagen crosslinking, chemical/thermal/biological/laser modification, gamma radiation, cryopreservation and so on. The said segment may be of varying geometry, varying thickness in any dimension, varying cross-sectional architecture, varying sizes in any dimension, varying radius and diameter and varying arc and/or chord length.

The foregoing description is for a few embodiments of the present invention. It should be appreciated that these embodiments are described for purpose of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

Other modifications will be apparent to those skilled in the art and, therefore, the invention is defined in the claims.

1-22. (canceled)

23. An implant for implantation into the cornea intra-stromally into a corneal channel or plane that comprises allogenic corneal or scleral material or other bioengineered material including but not limited to processed collagen wherein:
said implant is in the form of segment, ring or modified disc, either used as such or further modified through a variety of different ways, including but not confined to collagen crosslinking, chemical/thermal/biological/laser modification, gamma radiation, and/or cryopreservation, so as to modify shape, regional thickness variability, strength, stiffness, rigidity, safety, storage capabilities, drug elution and/or vector capabilities of the implant;
said implant changes or modifies among other characteristics, the shape and regularity of the patient's cornea, regularizes the conical cornea, gives an improved surface, improves biomechanical strength distribution and stability, improves optical functionality and improves/corrects the refractive error or gives other desired shape change effects;
said implant may be of varying geometry, varying regional thickness, varying thickness or geometry in any dimension or region, varying cross-sectional architecture, varying sizes in any dimension, varying radius and diameter and varying arc and/or chord length, and may be oval, rectangular, hexagonal, triangular, or other shape in cross-section and may have storage capabilities, drug elution and/or vector capabilities.

24. The implant of claim 23, further characterized:
in being a segment that is inserted into a corneal channel in order to obtain said desired effects;
said segment implant may be of varying geometry, varying regional thickness, varying thickness in any dimension, varying cross-sectional architecture, varying size in any dimension, varying radius and diameter, and varying arc and/or chord length, and may be oval, rectangular, hexagonal, triangular, or other shape in cross-section.

25. The implant of claim 23, further characterized:
in being an elongated segment that is inserted into a corneal channel in order to obtain said desired effects;
said elongated segment implant may be of varying geometry, varying regional thickness, varying thickness in any dimension, varying cross-sectional architecture, varying size in any dimension, varying radius and diameter, and varying arc and/or chord length, and may be oval, rectangular, hexagonal, triangular or other shape in cross-section.

26. The implant of claim 23, further characterized:
in being a ring that is inserted into a corneal channel or plane in order to obtain said desired effects;
said ring implant may be of varying geometry, varying regional thickness, varying thickness in any dimension, varying cross-sectional architecture, varying size in any dimension, varying radius and diameter, and varying arc and/or chord length, and may be oval, rectangular, hexagonal, triangular, or other shape in cross-section.

27. The implant of claim 23, further characterized:
in being a modified disc that is inserted into a corneal channel or plane in order to obtain said desired effects;
said modified disc implants may have regional variations in its geometry or thickness and may be of varying geometry, varying regional thickness, varying thickness in any dimension or region, varying cross-sectional architecture, varying sizes in any dimension, and varying radius and diameter.

28. The implant of claim 23, further characterized:
in being a segment, an elongated segment, a ring, or a modified disc that is further modified through a variety of different ways including but not confined to collagen crosslinking, chemical/thermal/biological/laser modification, gamma radiation, cryopreservation, so as to modify the shape, regional thickness variability, strength, stiffness, rigidity, safety, storage capabilities, drug elution or vector capabilities, said segment, elongated segment, ring, or modified disc implanted into a corneal channel or plane to obtain said desired effects;
said modified implants may have regional variations in its geometry or thickness, and may be of varying geometry, varying regional thickness, varying thickness in any dimension or region, varying cross-sectional architecture, varying size in any dimension, varying radius and diameter, and varying arc and/or chord length, and may be oval, rectangular, hexagonal, triangular, or any other shape in cross-section.
